(19) United States
(12) Plant Patent Application Publication
(10) Pub. No.: US 2006/0041977 P1
(43) Pub. Date: Feb. 23, 2006

(54) Double impatiens plant named 'Cameo Salmon Glow'

(75) Inventor: Xenia Cascante, Alajuela (CR)

Correspondence Address: THE WEBB LAW FIRM, P.C., 700 KOPPERS BUILDING, 436 SEVENTH AVENUE, PITTSBURGH, PA, 15219, US

(73) Assignee: Oglevee, Ltd., Connellsville, PA (US)

(21) Appl. No.: 11/209,362

(22) Filed: Aug. 23, 2005

Related U.S. Application Data
(60) Provisional application No. 60/603,780, filed on Aug. 23, 2004.

(51) Int. Cl. A01H 5/00

(52) U.S. Cl. PLT/317

(57) ABSTRACT

A new and distinct cultivar of Double Impatiens plant named ‘Cameo Salmon Glow’, characterized by its compact, mounded to semi-upright plant habit; rapid growth rate; freely branching habit; dense foliage, freely flowering habit; fully double flower form; and large salmon-colored flowers that are positioned above and beyond the foliage.

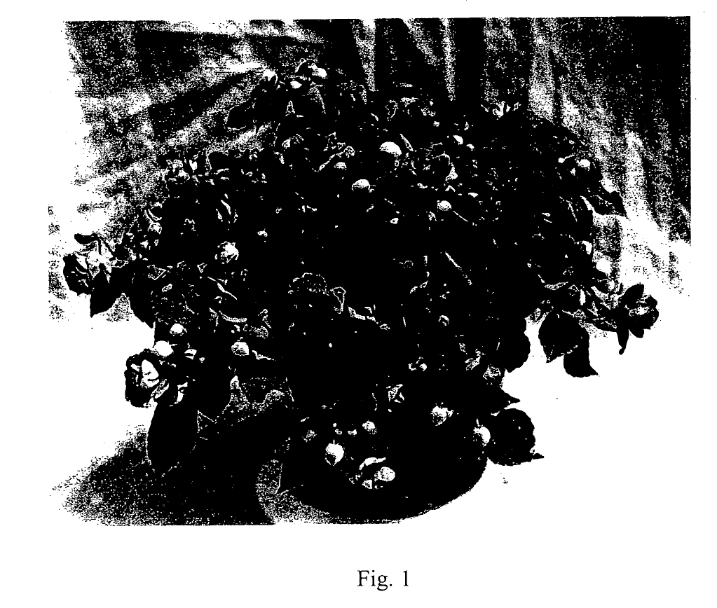

### CROSS-REFERENCE TO RELATED APPLICATIONS

This application claims the benefit of U.S. Provisional Plant Patent Application No. 60/603,780 filed Aug. 23, 2004, which is hereby incorporated by reference in its entirety.

### BOTANICAL CLASSIFICATION

Impatiens walleriana

### VARIETAL DENOMINATION

‘Cameo Salmon Glow’

### BACKGROUND OF THE INVENTION

The present invention comprises a new and distinct cultivar of Impatiens walleriana known by the varietal name ‘Cameo Salmon Glow’. The new variety is a product of a planned breeding program conducted in Cartago, Costa Rica. The purpose of the breeding program was to develop compact and mounded cultivars with fully double flowers, large flower size, and interesting flower colors. The new variety originated from a cross made of two proprietary selections of Impatiens walleriana: IDXBZW-1 (female parent) (unpatented) and IDXBZW-2 (male parent) (unpatented). Comparisons between the new variety and its parents cannot be made, as the parental varieties no longer exist. The cultivar ‘Cameo Salmon Glow’ was discovered and selected as a flowering plant within the progeny of the stated cross in a controlled environment in Cartago, Costa Rica. Asexual reproduction of the new cultivar by terminal cuttings has shown that the unique features of the new variety are stable and reproduced true to type in successive propagations by asexual reproduction.

The following traits have been repeatedly observed and are determined to be the unique characteristics of ‘Cameo Salmon Glow’. Characteristics that distinguish ‘Cameo Salmon Glow’ as a new and distinct cultivar from others known to the breeder include:

1) Large flower size of about 5.0 cm in diameter;

2) Compact, mounded to semi-upright growth habit;

3) Freely branching habit;

4) Rounded leaves with dense foliage;

5) Time to produce a finished plant in a 10 cm pot is 5 to 6 weeks; and

6) Salmon-colored flowers are carried above the leaf canopy and face upward.

Plants of the new variety can be compared to plants of the Impatiens walleriana cultivar ‘Cameo Salmon’, disclosed in U.S. Plant Pat. No. 12,313. In side-by-side comparisons conducted in Lompoc, Calif., plants of the new variety differed from plants of the cultivar ‘Cameo Salmon’ in the following characteristics:

1) Plants of the new variety had a mounded to semi-upright plant habit, whereas plants of the cultivar ‘Cameo Salmon’ had a mounded, spreading plant habit;

2) Plants of the new variety had flowers with a larger diameter than plants of the cultivar ‘Cameo Salmon’;

3) Plants of the new variety had a stem color of 147C with a cast of 184B, the cast being heaviest at the nodes, whereas plants of the cultivar ‘Cameo Salmon’ had a stem color of 146B;

4) Plants of the new variety had flowers with a color of 47C with streaks of 52C, whereas plants of the cultivar ‘Cameo Salmon’ had flowers with a color of 41D; and

5) Plants of the new variety had more petals per flower than plants of the cultivar ‘Cameo Salmon’.

### BRIEF DESCRIPTION OF THE DRAWING

The accompanying colored photographic drawing illustrates the new variety, with the colors being as nearly true as is possible to obtain in colored illustrations of this type.

### DESCRIPTION OF THE PLANT

The cultivar ‘Cameo Salmon Glow’ has not been observed under all possible environmental conditions. The phenotype may vary somewhat with variations in environment such as temperature, day length and light intensity. However, there is no variance in genotype.

In the following description, color references are made to The Royal Horticultural Society Colour Chart, 1995 Edition, except where general terms of ordinary dictionary significance are used. Plants were grown under commercial practice during the late winter through early spring of 2003 in Lompoc, Calif. in a corrugated polycarbonate-covered greenhouse with 30% shade covering. During the production of the plants, day temperatures ranged from 21 to 24° C.; night temperatures ranged from 16 to 18° C.; and light levels ranged from 4000 to 5000 foot candles of light. Plants used in the following description were grown in 15 cm containers for about nine to ten weeks after planting rooted cuttings.

### Propagation

Type cutting: Terminal cuttings.
Time to initiate roots:
   Summer.—About 10 days at 21° C.
   Winter.—About 12 days at 21° C.
Time to produce a rooted cutting:
   Summer.—About 21 days at 21° C.
   Winter.—About 21 days at 21° C.
Root description: Fibrous, fine, white in color.
Root habit: Freely branching, dense.

### Plant Description

General appearance: Compact and mounded to semi-upright. Appropriate for 10-15 cm pots and 20 to 25 cm hanging basket containers.
Growth and branching habit: Vigorous and freely-branching habit with at least 10 lateral branches developing per plant, dense growth. Pinching (removal of terminal apex) is usually not required.
Crop time: Relatively rapid growth rate.
   From planting one rooted cutting in a 10 cm pot.—About 5 to 6 weeks.
   From planting three rooted cuttings in a 25 cm hanging basket container.—About 8 to 10 weeks.
Plant height at flowering: About 18 to 21 cm.
Lateral branch description:
   Length.—About 18 to 21 cm.
   Diameter.—About 8 to 10 mm.
   Internode length.—About 1 to 3 cm.
   Texture.—Smooth, glabrous.
   Color.—147C with a cast of 184C, the cast being heaviest at the nodes.
Foliage description:
   Arrangement.—Alternate, simple; densely foliated.
   Length.—About 5 to 5.5 cm.
   Width.—About 2.5 to 3 cm.
   Shape.—Ovate.
   Apex.—Acute.
   Base.—Acute.
   Margin.—Serrated with fine ciliation.
   Texture, upper and lower surfaces.—Glabrous.
   Venation pattern.—Pinnate.
   Color.—Developing leaves, upper surface: 137A. Developing leaves, lower surface: 147C. Fully expanded leaves, upper surface: 137B. Fully expanded leaves, lower surface: 148B. Venation, upper surface: 147C. Venation, lower surface: 137A.
   Petiole.—Length: About 8 to 10 mm. Diameter: About 3 mm. Texture upper and lower surface: Smooth, glabrous. Color: 146C with a slight cast of 184C.

### Flower Description

Flower type and habit: Numerous and consistently double salmon-colored flowers. Freely and continuously flowering. Flower buds open similar to a rose in fullness; flowers rounded. Usually 8 to 15 flowers and flower buds per lateral branch. Flowers positioned above the foliage and typically face outward to upward. Flowers last about 7 to 10 days on the plant. Flowers not persistent. Flowers not fragrant.
Flowering season: Year-round under greenhouse conditions. In the garden, flowering from spring until fall. Flowering indeterminate and continuous.
Flower diameter: About 4.5 to 5 cm.
Flower depth (height): About 2 cm.
Flower buds:
   Rate of opening.—From full color bud to full open flower, about 5 to 7 days is typical depending on temperature.
   Length.—About 15 mm.
   Diameter.—About 12 mm.
   Shape.—Almost round.
   Color (just before opening).—47B.
Petals:
   Quantity/arrangement.—About 30 to 35; imbricate.
   Length, outer petals.—About 20 to 22 mm.
   Width, outer petals.—About 18 to 20 mm.
   Shape.—Cordate.
   Apex.—Rounded with most petals showing indentations.
   Base.—Acute.
   Margin.—Entire.
   Texture, upper and lower surfaces.—Glabrous, smooth.
   Color.—When opening, upper surface: 47B with streaks of 54A. When opening, lower surface: 54A with a cast of 41C around the mid rib. Fully opened, upper surface: 47C fading to 51A with streaks of 52C. Fully opened, lower surface: 52C with a cast of 41C around the mid rib.
Peduncles:
   Length.—About 30 to 35 mm.
   Diameter.—About 2 mm.
   Angle.—About 45° to the lateral branch.
   Strength.—Strong.
   Texture.—Smooth, glabrous.
   Color.—147C with a cast of 184D.
Reproductive organs: Not applicable due to full double flowers.
Fruit/seed development: Fruit and seed development has not been observed.
Disease/Pest resistance: Plants of the new Impatiens have not been observed to date to be resistant to pathogens and pests common to Impatiens.
Temperature tolerance: Plants of the new Impatiens have been observed to be tolerant to rain, wind, and temperatures ranging from 5 to 38° C.

I claim:

1. A new and distinct variety of Impatiens named ‘Cameo Salmon Glow’ as herein described and illustrated
